(19) United States
(12) Patent Application Publication
(10) Pub. No.: US 2014/0257478 A1
(43) Pub. Date: Sep. 11, 2014

(54) ACCOMMODATING FLUIDIC INTRAOCULAR LENS WITH FLEXIBLE INTERIOR MEMBRANE

(71) Applicant: Sean J. McCafferty, Tucson, AZ (US)

(72) Inventor: Sean J. McCafferty, Tucson, AZ (US)

(21) Appl. No.: 14/195,345

(22) Filed: Mar. 3, 2014

Related U.S. Application Data
(63) Continuation-in-part of application No. 14/193,301, filed on Feb. 28, 2014.
(60) Provisional application No. 61/773,909, filed on Mar. 7, 2013.
(60) Provisional application No. 61/775,752, filed on Mar. 11, 2013.
(60) Provisional application No. 61/775,752, filed on Mar. 11, 2013.

(51) Int. Cl. A61F 2/16

(52) U.S. Cl. CPC A61F 2/1637
USPC 623/6.13

(57) ABSTRACT

An accommodating (re-focusable) intraocular lens (IOL), a body of which includes two optical portions sequentially disposed, in optical contact with one another, along an optical axis and separated by interior surface the curvature of which is changing in response to pressure applied to posterior surface of IOL. The two optical portions may be formed with fluids having different refractive indices and housed in flexible cells that share an interior wall having such interior surface. The wall bends or flexes in response to force, caused by flexing of ciliary body muscle when IOL is installed in eye's capsule's membrane and passed onto the body of IOL via bendable haptics integrated with the optical portion(s) along a perimeter. The optical power of the IOL is gradually modifiable in part due to change of curvature of the interior surface.

### CROSS-REFERENCE TO RELATED APPLICATIONS

The present U.S. Patent application claims priority from and benefit of the U.S. Provisional Patent Applications Nos. 61/773,909 filed on Mar. 7, 2013 and titled “Fluidic Membrane Accommodating Intraocular Lens” and 61/775,752 filed on Mar. 11, 2013 and titled “Aspheric Intraocular Lens With Continuously Variable Focal Length.” The present patent application is also a continuation-in-part from the U.S. patent application Ser. No. 14/193,301 filed on Feb. 28, 2014, and titled “Refocusable Intraocular Lens With Flexible Aspherical Surface” (attorney docket 147923.00010), which in turn claims priority from U.S. Provisional Patent Application 61/775,752. The disclosure of each of the above-mentioned patent documents is incorporated herein by reference in its entirety.

### TECHNICAL FIELD

The present invention relates to ophthalmological instruments and, more particularly, to an intraocular lens having a posterior aspheric surface with mechanically-modifiable curvature and a continuously alterable focal length.

### BRIEF DESCRIPTION OF THE DRAWINGS

The invention will be more fully understood by referring to the following Detailed Description in conjunction with the generally not-to-scale Drawings, of which:

FIG. 1A is a diagram showing, in front view, an embodiment of the intraocular lens of the invention;

FIG. 1B is a cross-sectional perspective view of the embodiment of FIG. 1A;

FIG. 2A is a diagram of a human eye;

FIG. 2B is a diagram illustrating an example of operable placement of the embodiment of FIGS. 1A, 1B in a human eye;

FIG. 2C is a diagram illustrating another example of operable placement of the embodiment of FIGS. 1A, 1B in a human eye;

FIG. 3 shows an alternative embodiment of the intraocular lens of the invention;

FIG. 4 shows another alternative embodiment of the intraocular lens of the invention;

FIGS. 5A, 5B illustrate layouts of a model of the human eye with the pseudophakic lens of the invention placed therein in Zemax® optical modeling software, showing the shape change of the front and back surface of the lens to alter the eye's focal distance from infinity to near;

FIGS. 6A, 6B present spot diagrams generated in Zemax® and corresponding, respectively, to layouts of FIGS. 5A, 5B;

FIGS. 7A, 7B show images of the same object formed with an embodiment of the invention accommodated according to the layouts of FIGS. 5A, 5B;

FIG. 8 is a flow-chart schematically depicting a method according to an embodiment of the invention;

FIGS. 9A and 9B are diagrams showing, in front and cross-sectional perspective view, an alternative embodiment of the invention;

FIG. 10 is a diagram illustrating an example of operable placement of the embodiment of FIGS. 9A, 9B in a human eye;

FIGS. 11A, 11B illustrate layouts of a model of the human eye with the pseudophakic lens of the invention placed therein in Zemax® optical modeling software, showing accommodation of the lens to alter the eye's focal distance from infinity to a near position;

FIGS. 12A, 12B present spot diagrams generated in Zemax® and corresponding, respectively, to layouts of FIGS. 11A, 11B;

FIGS. 13A, 13B show images of the same object formed with an embodiment of the invention accommodated according to the layouts of FIGS. 11A, 11B.

### SUMMARY

Embodiments of the invention provide a pseudophakic lens that includes a first rotationally symmetric optical portion having an optical axis and a first optical power, a second rotationally-symmetric optical portion co-axial with the first rotationally symmetric portion and having a second optical power, and first and second flexible haptic wings, each having proximal and distal sides. A first volume, being the volume of the first rotationally symmetric optical portion is defined by a posterior curved plate having a first perimeter and a flexible membrane, the first volume is filled with a first fluid having a first refractive index. A second volume—being the volume of the second rotationally-symmetric optical portion, is defined by an anterior rigid curved plate having a second perimeter and the flexible membrane. The second volume is filled with a second fluid having a second refractive index. The posterior and anterior plates are integrated with one another along said first and second perimeters. The flexible and deformable membrane is sealingly affixed to at least one of the posterior and anterior plates at least one of the first and second perimeters such as to prevent leakage or escape of any of the first and second fluids from a respectively corresponding volume of the first and second volumes. The proximal sides of the first and second haptic wings are integrated with at least the anterior plate at least along the first perimeter. The first and second optical portions are structured to be operable such as to gradually change at least one of the first and second optical powers in response to deformation of the flexible and deformable membrane while the anterior and posterior plates substantially maintain their corresponding shapes.

The presudophakis lens is dimensioned to be placed, in operation, in mechanical cooperation with a ciliary body muscle of an eye of a subject such that, in response to tension applied to at least one of zonules and capsular membrane of a natural lens of the eye by the ciliary body muscle, an anteriorly-vectored force is administered to said posterior plate, causing deformation of the flexible membrane by transferring of pressure thereto from the posterior plate through the second fluid. The deformation of the membrane can be spherical. In a specific implementation, the lens may additionally include a rotationally symmetric stabilizing plate made from an optically transparent material. Such stabilizing plate has a surface congruent with that of the posterior plate and is integrated with said posterior plate along an outer surface thereof.

Embodiments additionally provide a pseudophakic lens having an optical power and including a bicameral chamber defined by rigid and foldable anterior and posterior curved layers of material integrated with one another along corresponding perimeters of such layers. This embodiment also includes a flexible and deformable membrane disposed between the anterior and posterior layers to form first and second cameras or sub-chambers, of said chamber, filled respectively with first and second fluids that have different indices of refraction. The flexible membrane is sealingly and directly affixed to said corresponding perimeters to prevent leakage of any of said first and second fluids from corresponding sub-chambers. The lens is structured to be operable to transfer pressure, applied anteriorly to the posterior layer, to the membrane such as to change the optical power in response to spherical deformation of membrane caused by such pressure transfer transfer. In a specific case, the lens is operable to change the optical power in response to such spherical deformation while the deformation is accompanied by at least one of (i) the anterior and posterior layers substantially maintaining their corresponding shapes, and (ii) the anterior and posterior layers substantially maintaining their corresponding axial positions. Additionally or alternatively, the posterior plate in unstressed state has a prolate aspheric shape.

Embodiments of the invention additionally provide a method for correcting vision with the use of an intraocular lens (IOL). Such method includes a step of implanting the IOL in an eye of the patient. The IOL as issue has a bicameral chamber defined by rigid and foldable anterior and posterior curved layers of material integrated with one another along corresponding perimeters of such anterior and posterior curved layer. The IOL also has a flexible and deformable membrane disposed between the anterior and posterior layers such as to form first and second cameras or sub-chambers, of said chamber, that are filled respectively with first and second fluids having different indices of refraction. The IOL also includes first and second flexible haptic wings, each having proximal and distal sides. The proximal sides of the haptic wings are integrated with at least the anterior layer at least along a perimeter of this layer. The flexible and deformable membrane is sealingly and directly affixed to said corresponding perimeters to prevent leakage of any of said first and second fluids from corresponding sub-chambers. The lens is operable to transfer pressure (when it's applied to the posterior layer towards the front of the lens), to the membrane such as to change the optical power in response to spherical deformation of membrane caused by such pressure transfer.

The method additionally includes a step of juxtaposing the haptic wings and the posterior layer against an interior surface of a capsule membrane of a natural lens of the eye such as to place distal side of each of the haptic wings in mechanical cooperation with said capsule membrane. Alternatively or in addition, the method may include a step of spherically deforming the flexible membrane by applying, to the posterior layer, force that is directed anteriorly (towards the anterior layer).

### DETAILED DESCRIPTION

The clouding of the natural lens of an eye, which is often age-related, is referred to as cataract. Visual loss, caused by the cataract, occurs because opacification of the lens obstructs light from traversing the lens and being properly focused on to the retina. The cataract causes progressive decreased vision along with a progressive decrease in the individual's ability to function in his daily activities. This decrease in function with time can become quite severe, and may lead to blindness. The cataract is the most common cause of blindness worldwide and is conventionally treated with cataract surgery, which has been the most common type of surgery in the United States for more than 30 years and the frequency of use of which is increasing. As a result of cataract surgery, the opacified, clouded natural crystalline lens of an eye is removed and replaced with a synthetic and clear, optically transparent substitute lens (often referred to as an intraocular lens or IOL) to restore the vision.

The use of such customized synthetic IOLs that are properly sized for a given individual—often referred to as intraocular lenses—has been proven very successful at restoring vision for a predetermined, fixed focal distance. The most common type of IOL for cataract treatment is known as pseudophakic IOL that is used to replace the clouded over crystalline lens. (Another type of IOL, more commonly known as a phakic intraocular lens (PIOL), is a lens which is placed over the existing natural lens used in refractive surgery to change the eye's optical power as a treatment for myopia or nearsightedness.) An IOL usually includes of a small plastic lens with plastic side struts (referred to as “haptics”), which hold the IOL in place within the capsular bag inside the eye. IOLs were traditionally made of an inflexible material (such as PMMA, for example), although this is being superseded by the use of flexible materials. Such lenses, however, are not adapted to restore the eye's ability to accommodate, as most IOLs fitted to an individual patient today are monofocal lenses that are matched to “distance vision”.

Accommodation is the eye's natural ability to change the shape of its lens and thereby change the lens' focal distance. The accommodation of the eye allows an individual to focus on an object at any given distance within the field-of-view (FOV) with a feedback response of an autonomic nervous system. Accommodation of an eye occurs unconsciously, without thinking, by innervating a ciliary body muscle in the eye. The ciliary muscle adjusts radial tension on the natural lens and changes the lens' curvature which, in turn, adjusts the focal distance of the eye's lens.

Without the ability to accommodate one's eye, a person has to rely on auxiliary, external lenses (such as those used in reading glasses, for example) to focus his vision on desired objects. Typically, cataract surgery will leave an individual with a substantially fixed focal distance, usually greater than 20 feet. This allows the individual to participate in critical activities, such as driving, without using glasses. For activities such as computer work or reading (which require accommodation of eye(s) at much shorter distance), the individual then needs a separate pair of glasses.

Several attempts have been made to restore eye accommodation as corollary to cataract surgery. The most successful of used methodologies relies on using a substitute lens that has two or three discrete focal lengths to provide a patient with limited visual accommodation in that optimized viewing is provided at discrete distances—optionally, both for distance vision and near vision. Such IOLs are sometimes referred to as a “multifocal IOLs”. The practical result of using such IOLs has been fair, but the design compromises the overall quality of vision. Indeed, such multifocal IOLs use a biconvex lens combined with a Fresnel prism to create two or more discreet focal distances. The focal distance to be utilized is in focus while there is a superimposed defocused image from the other focal distances inherent in the lens. Also, the Fresnel prism contains a series of imperfect dielectrical boundary-related discontinuities, which create scatter perceived as glare by the patient. Some patients report glare and halos at night time with these lenses.

Another methodology may employ altering the position of a fixed-focal-length substitute lens (often referred to as an “accommodating IOL”) with contraction of a ciliary muscle to achieve a change in the working distance of the eye. These “accommodating IOLs” interact with ciliary muscles and zonules, using hinges at both ends to “latch on” and move forward and backward inside the eye using the same natural accommodation mechanism. In other words, while the fixed focal length of such IOL does not change in operation, the focal point of an “accommodating IOL” is repositioned (due to a back-and-forth movement of the IOL itself) thereby changing the working distance between the retina and the IOL and, effectively, changing the working distance of the IOL. Such IOL typically has an approximately 4.5-mm square-edged optical portion and a long hinged plate design with polyimide loops at the end of the haptics. The hinges are made of an advanced silicone (such as BioSil). While “accommodating IOLs” have the potential to eliminate or reduce the dependence on glasses after cataract surgery and, for some, may be a better alternative to refractive lens exchange (RLE) and monovision, this design has diminished in popularity due to poor performance and dynamic range of movement that is not sufficient for proper physiological performance of the eye.

Therefore, there remains an unresolved need in an IOL that is structured to be, in operation, continuously accommodating, with gradually, non-discretely and/or monotonically adjustable focal length.

According to an embodiment of the invention, the problem of accommodating the focal length of an IOL is solved by utilizing a force mechanism supplied by the eye's ciliary muscle. The IOL is provided with a flexible aspherical surface and is juxtaposed in such spatial relation with respect to the ciliary muscle that force, transferred to the IOL by the muscle, applies pressure on the posterior surface of the accommodating IOL to changes the curvature of the posterior surface and, thereby, the power of the IOL as well. Specifically, according to an idea of the invention, an embodiment of the accommodating IOL is structured to utilize, when implanted into an eye, gradually-changing radial tension caused by the relaxing ciliary muscle thus creating an anteriorly-directed force applied to alter the posterior curvature of the IOL and, as a result, the overall lens' power. The change in radial tension associated with the implanted IOL enables the patient who has undergone cataract surgery to gradually vary the focal length of the IOL through the eye's natural mechanism of ciliary body muscle tension, i.e. in substantially the same way as the focal length of the natural, crystalline lens of an eye is varied. Such variation of the focal length is achieved without repositioning of the IOL itself.

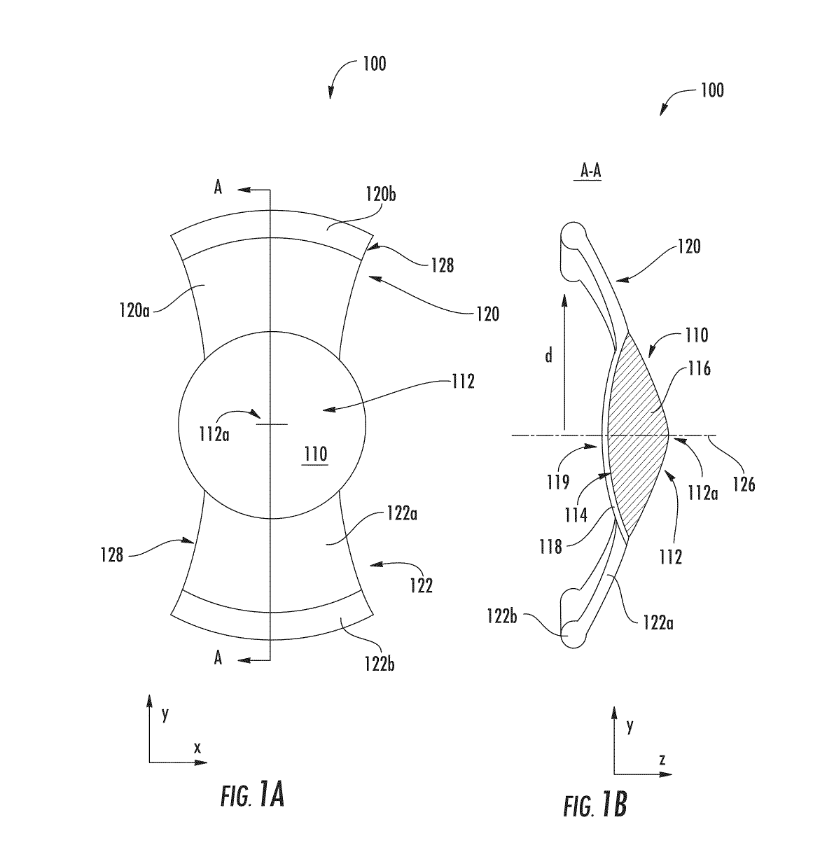

FIG. 1A is a diagram showing an embodiment 100 of the IOL according to the invention in front view, while FIG. 1B displays a cross-sectional perspective view of the embodiment 100. The local system of coordinates is chosen such that the z-axis generally corresponds to a direction of ambient light propagation through the IOL that has been implanted in the eye. The embodiment 100 includes an optical portion 110 containing a first lenticle or lenslet 116 such as an axially-symmetric aspheric lens having a posterior surface or boundary 112 (in one example—a prolate aspheric surface) and an anteriorly disposed surface or boundary 114 (in one example—an oblate aspheric surface). The boundary surfaces 112, 114 defines a volume of the lenslet 116 filled with biocompatible material such as gel-silicone or sylgard®, for example.

The optical portion may be optionally enhanced and complemented with a stabilizing plate 118 (made, for example, with Acrylic) disposed in front of the first lenticle 116 (as viewed from the apex 112a of the anterior surface 112) such as to share an optical interface 114 with the first lenticle 116. The plate 118 is defined by the anteriorly intermediate surface 114, which it shared with the first lenticle 116, and a front outer or posterior surface 119. It is appreciated, that in a specific implementation and depending on the curvatures of the surfaces 114, 119, the stabilizing plate 118 may be structured as a second lenticle or lenslet 118 disposed in front of the first lenticle 116. The elements 116, 118 aggregately define an optical portion 110 of the IOL 100.

As shown, both the first lenslet 116 and the plate 118 are radially extended, on the outboard side of the optical portion 110, by at least two haptics 120, 122 that are interconnected by the stabilizing plate 118. In the embodiment 100, the haptics 120, 122 are shown integrated with the plate 118 and, in particular, with the front outer surface 119 such as to form a spatially-continuous structure formed by the elements 120, 118, 122. This spatially-continuous structure, which carries the lenslet 116, is configured as a lenslet 116 supporting structure that contains a central optical portion 118 and the haptic wings 120, 122. In one implementation the haptics are symmetric about an optical axis 126 of the lenticle 116. In a related implementation (not shown in FIGS. 1A, 1B), the haptics may include an odd number of haptic wings that may be disposed asymmetrically with respect to the optical axis 126 (z-axis in FIG. 1B). The haptics include substantially spatially continuous wing portions 120a, 122a and may optionally include peripheral ridge portions (interchangeably referred to herein as ridges) 120b, 122b characterized by increased thickness and/or rounded edges as compared to the wings 120a, 122a and connected by the wings 120a, 120b with the central optical portion 110, 118. Furthermore, the haptics and contiguous anterior lens surface are a relatively rigid structure when compared to the more pliable posterior lenticle which changes its surface shape in order to actuate the accommodation utilizing the net anterior vectored force supplied by natural tightening zonules in physiologic accommodation. The haptics are designed to be supported in their rigidity within the natural capsule retained following cataract extraction. The haptic design is such that it conforms to the posterior surface of the capsule out to its equator and thereby is able to counter the net anterior vectored force by transmitting the force centripetally to the equator of the capsule. Lastly the haptics are designed to a width so as to increase rigidity and prevent rotational buckling. The, outer limits of the haptics are flared with rounded edges to distribute stress over a large area in the capsule which limits non-azimuthally symmetric deformation and the risk of capsular rupture.

In further reference to FIGS. 1A, 1B, in one embodiment each of the anterior lenslet 116, plate 118, and/or the wings of haptics 120a, 122a is substantially materially homogeneous and devoid of discontinuities in shape and/or refractive index. Such homogeneity and continuity of shape enables reduction of light glare due to light scatter on a surface of the embodiment 100 and/or optical aberrations caused by diffraction of light on discontinuities upon light traversal of the embodiment 100. In one embodiment, the plate 118 (which may be structured as a second or posterior lenslet 118, as mentioned above) is formed from the same material (for example, acrylic) and is integral with (for example, co-molded) the haptics 120, 122. In a related embodiment, the posterior lenticle 118 is optionally made from a highly flexible material (such as silicone gel, Slygard 184) with memory fused to a much stiffer anterior surface 112.

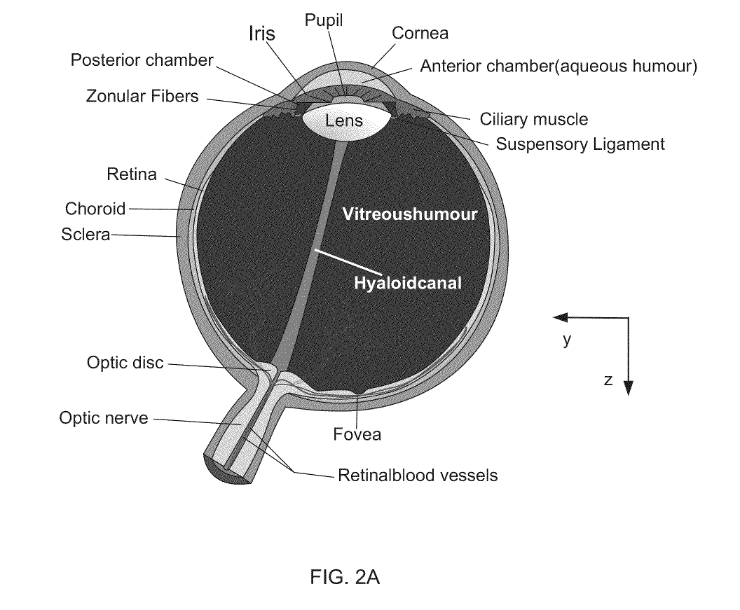

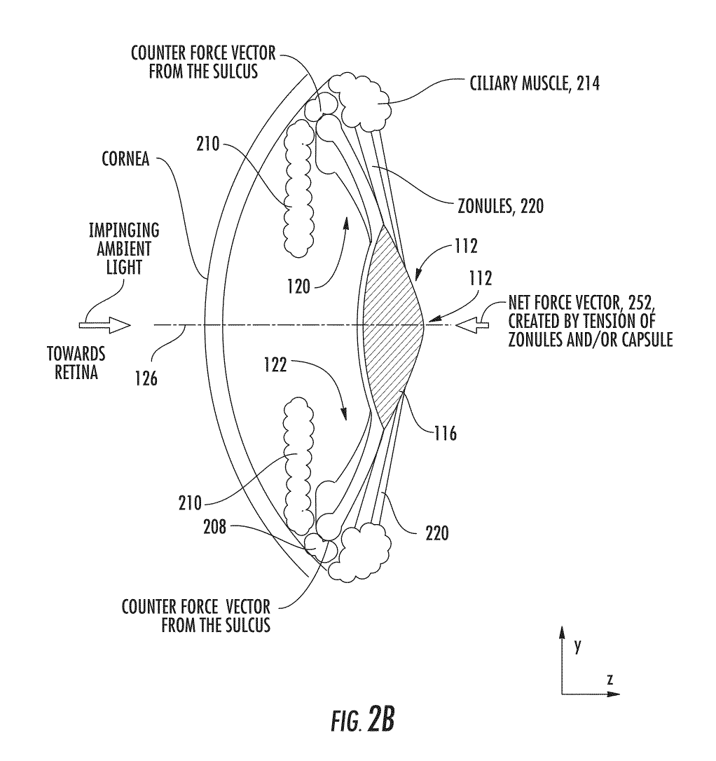

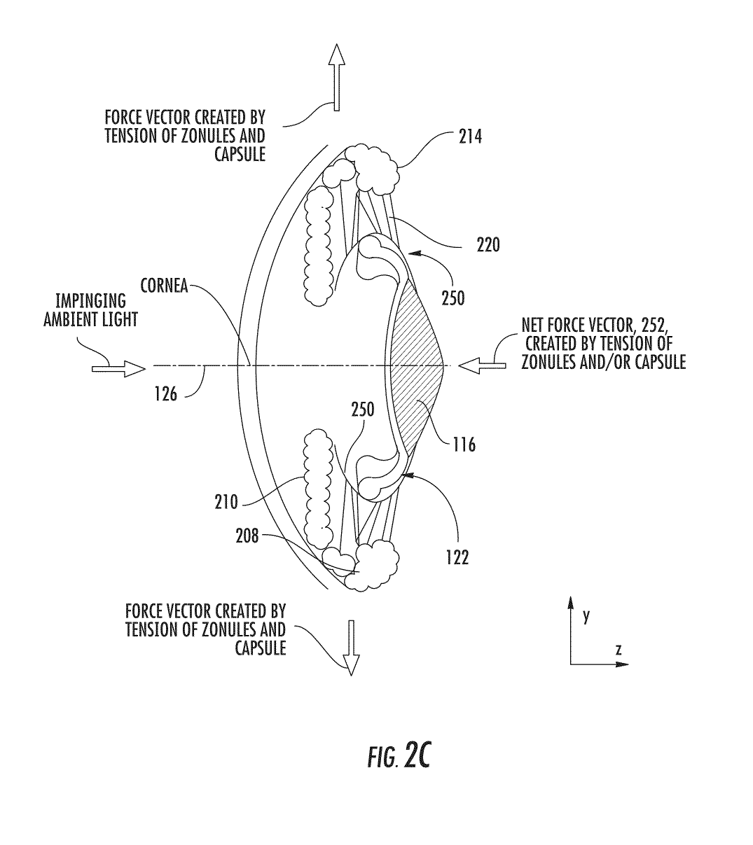

FIG. 2A shows diagrammatically the human eye. In reference to FIG. 2A, FIGS. 2B and 2C illustrate, in simplified cross-sectional views, examples of operable cooperation with and spatial orientation of the embodiment 100 inside the eye.

As shown in FIG. 2B, in operation, the outmost portions of haptics (such as ridges 120b, 122b) of the embodiment of the IOL of the invention may be placed in the sulcus 208 of the eye (the groove, crevice, furrow, or space formed between the root of the iris 210 and the ciliary body muscle 214) such that the wings 120a, 122a are positioned in front of the zonules 220. The zonules abut the equator of the lens capsule that is under tension. The zonules are under tension provided by abutted pressure supplied by the haptics. The unstressed shape of a posterior surface (114 and/or 119) of the optical portion of the embodiment of the invention is substantially that of a prolate (a)sphere. As shown schematically in FIG. 2C, the outmost portions of haptics (for example, ridges 120b, 122b are placed in the capsule 250 of the now-removed natural lens of the eye to be abutted against the anterior equator of the capsule 250. When the ciliary body muscle 214 is relaxing (for example, during the focusing of the eye at a large distance), tension on the zonules (ciliary zonules) 220 and/or the capsule 250 is increased centripetally and, as a result, the surface 112 is being tightened. The details of the deformation of the lenslet 116 are further shown and discussed below in reference to FIG. 2B (although a similarly operable deformation occurs in case when the embodiment 110 is disposed according to FIG. 2A)

The centripetal tightening in the x-y plane of both the zonules 220 and/or the capsule 250which have been placed under slight tonic tension by the IOL/haptics displacing the capsule posteriorly in the +z direction. The conical displacement of the capsule 250 and zonules 220 with its apex in the +z direction (posteriourly) causes any additional centripetal tension supplied by relaxation of the ciliary muscle 214 provides pressure, through the zonules and capsule, to the deformable surface 112 of the IOL 110. The net vector of this applied pressure, shown in FIGS. 2B, 2C with an arrow 252, forms a force in the −z direction. The abutted haptics provide a counter force in the +z direction to prevent the lens from translating in the z axis. This net +z force is translated by the curved haptics abutted against the capsule 250 to internal tension within the capsule in the x-y plane. The pressure in the −z direction supplied by the tension of the zonules 220 and capsule 250 (which acts as a membrane in contact with the IOL surface 112) will be unequally distributed across the surface inversely proportional to its radius of curvature. Stated differently, pressure is supplied by the tension of the overlying membrane preferentially to the apex of the prolate aspherical surface 112, thus flattening this aspherical surface. Overall, there is an increase in the radius of curvature of surface 112 with increased tension, which allows the IOL 100 to (re)focus at distance in a natural physiological manner. It is appreciated that the strength of the anterior pressure and, therefore, the amount of anterior force is substantially directly proportional to the posterior displacement of the lenslet 116. Therefore, the higher pressure is applied to the central portion (including the apex 112a and the immediately surrounding areas) of the prolate aspheric surface 112 than to its peripheral annular portion circumscribing the central portion. The pressure differential experienced by the central portion and the peripheral portion of the surface 112 and caused by the relaxation of the ciliary body muscle 214 compels a change of curvature (and, in particular, flattening) of the aspheric surface 112 thereby reducing the overall power of the optical portion of the IOL 100 in a fashion substantially similar to that causing the reduction of the natural crystalline lens of the eye during relaxation of the eye to accommodate the vision on a distant object.

Consequently to flattening of the surface 112, optical imaging conditions are formed that correspond to a distant object within the FOV of the IOL 100 becoming an optical conjugate of the retina (not shown in FIGS. 2B, 2C). As the degree of flattening of the surface 112 and, therefore, a reduction of optical power of the lenticle 110 depends on the gradually and continuously varying degree of relaxation of the ciliary muscle 214, the accommodation of the vision at a distance is also gradual and continuous.

During the contraction of the ciliary muscle 214, on the other hand, the tension on the zonules 220 and the membrane of the capsule 250 is being reduced, thereby causing decrease in pressure on the posterior surface 112 and restoring the posterior surface 112 from its flattened condition towards a more curved one and towards that of a prolate asphere corresponding to the relaxed condition of the muscle 214. As a result, the overall power of the optical portion 110 of the IOL 100 is increased, thereby defining the retina and a near-by object located within the FOV of the IOL 100 as optical conjugates. As the degree of steepening of the curvature of the surface 112 and, therefore, increase of the optical power of the lenticle 110 depend on the gradually and continuously varying degree of contraction of the ciliary muscle 214, the accommodation of the vision at near-by objects is also gradual and continuous.

Accommodation of the vision on near-by objects is accompanied with miosis (pupilary constriction). Embodiments of the IOL of the invention are structured to take advantage of this physiological process. With constriction of the pupil and during the optical accommodation of the embodiment of the IOL, the optical performance of the IOL is substantially restricted to the area of the optical portion of the IOL that is located centrally and that is adjacent to the apex 112a of the lenslet 110, because the clear optical aperture defined by the pupil is being reduced in size. As the curvature of the prolate aspheric surface 112 in its central, neighboring the apex 112a portion is higher than in any other portion of the surface 112, the change in the overall resulting optical power of the IOL 100 achieved due to the accommodating of the ciliary muscle 214 during the miosis is larger than during a period of time when the pupil of the eye is not constricted.

Referring again to FIG. 1B and in further reference to FIGS. 2B and 2C, the front outer (most anterior) surface 119 of the IOL 100 is shaped as an oblate asphere that has a lower degree of asphericity and curvature of the opposite sign as compared with those of the posterior surface 112. As a result, spherical aberrations that are caused by the posterior surface 112 (while transmitting ambient light that emanates from a distant object within the FOV of the IOL 100 to the object's conjugate at the retina during the period of time when the pupil is dilated) are at least partially compensated. The (slightly larger central radius of curvature) in surface 119 (in comparison with the surface 112, which has a much smaller central radius of curvature, also facilitates, in combination with the miotic pupil, taking operational advantage of the prolate posterior surface 112 (which also increases the lens) power during accommodation.

It is worth noting that one operational shortcoming of (other) mechanical structures of accommodating IOLs of the related art is that the small force applied by the capsule 116 has to be sufficient to actuate the lens and alter its shape and power. (The small actuating/accommodating force of about 1 gram is applied most effectively to the present design as opposed to other designs). In contradistinction with accommodating IOLs of the related art, embodiments of the present invention are structured to directly transfer the force, caused by flexing of the ciliary body muscle, to a posterior surface 112 of the optical portion of the embodiment to alter its shape, causing substantially no loss of force upon transmission that would otherwise occur if the force were transferred to any other an internal or anterior surface of the optical portion of the embodiment.

It will be understood by those of ordinary skill in the art that modifications to, and variations of, the illustrated embodiments may be made without departing from the inventive concepts disclosed in this application. For example, in reference to FIGS. 1A, 1B, while in general the shapes of the wing portions 120a, 122b of the haptics may vary, it may be preferred that the wing portions 120a, 122a be curved in at least one of a meridian plane that contains an optical axis (such as the yz-plane, for example) and an azimuthal plane (such as the xz-plane), such that a given wing of a haptic forms a portion of a dome and, in one embodiment, conforms to the natural shape of the natural lens of the eye such as to maintain the capsule 250 in its physiological shape when placed therein. For example, a given haptic (such as the haptic 120 of FIGS. 1A, 1B) may be curved radially (in yz-plane) or azimuthally (in xz-plane). Alternatively, at least one haptic can be curved in two planes that are transverse to one another (for example, a haptic may have a surface that is curve both radially and azimuthally). In one specific example, an embodiment of the IOL of the invention includes multiple haptics that are portions of the spherical sector defined by the haptics with respect to a center of curvature of a haptic. The ridges of individual haptics may lie on the same circle. The side boundaries of the haptics (such as boundaries 128 in front view of FIG. 1A) may be defined by straight lines or curved lines.

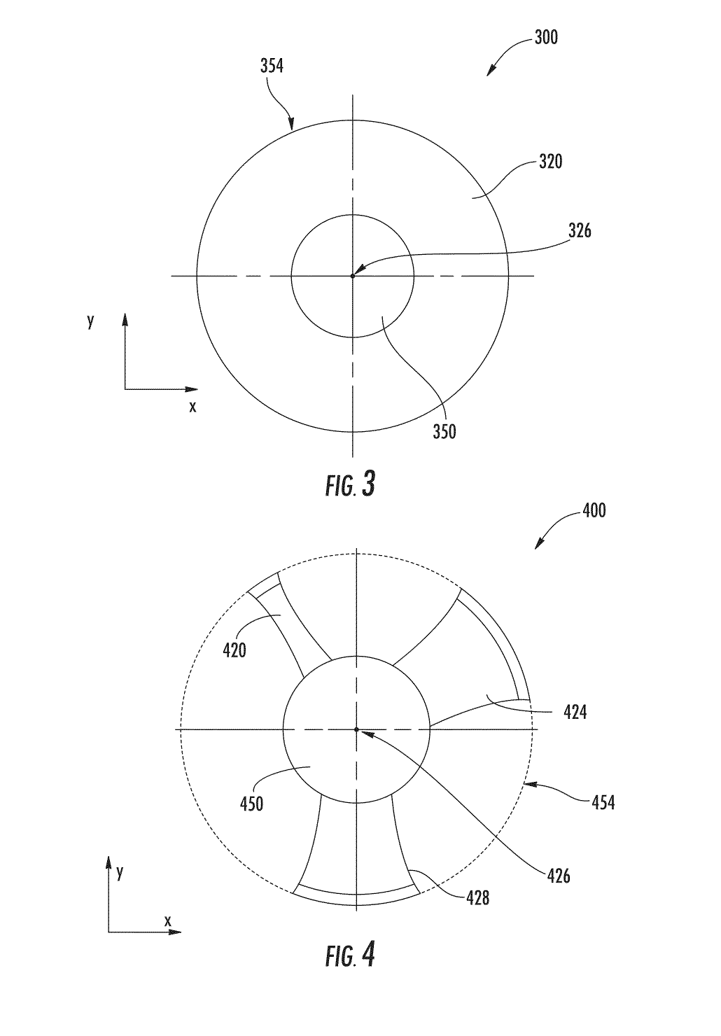

FIGS. 3 and 4 show, in front views, alternative embodiments 300, 400 of the IOL according to the invention. The embodiment 300 boasts a structure that is substantially rotationally symmetric with respect to the axis 326 and that includes a single haptic 320, without a ridge portion, that forms a peripheral skirt around the perimeter of the lenslet portion 350. The embodiment 400 illustrates an IOL structure containing three haptics 420, 424, 428 that are sized differently and disposed asymmetrically with respect to the optical axis 426 of the optical portion 450. While in both embodiments 300, 400 lines 354, 454 (on which the outer perimeters of the corresponding haptics 320 and 420, 424, 428 lie) are shown to form a circle in a plane that is substantially perpendicular to the axes 326, 426, generally the radial separations (such as the distance d of FIG. 1A, 1B) between perimeter line(s) of different haptics and the axis of the corresponding optical portion of a given embodiment may vary. A related embodiment (not shown) may be devoid of the stabilizing plate 118 and the haptics 120, 122 may be directly molded to the optical portion 110 to form flexible peripheral flanges with respect to the portion 110.

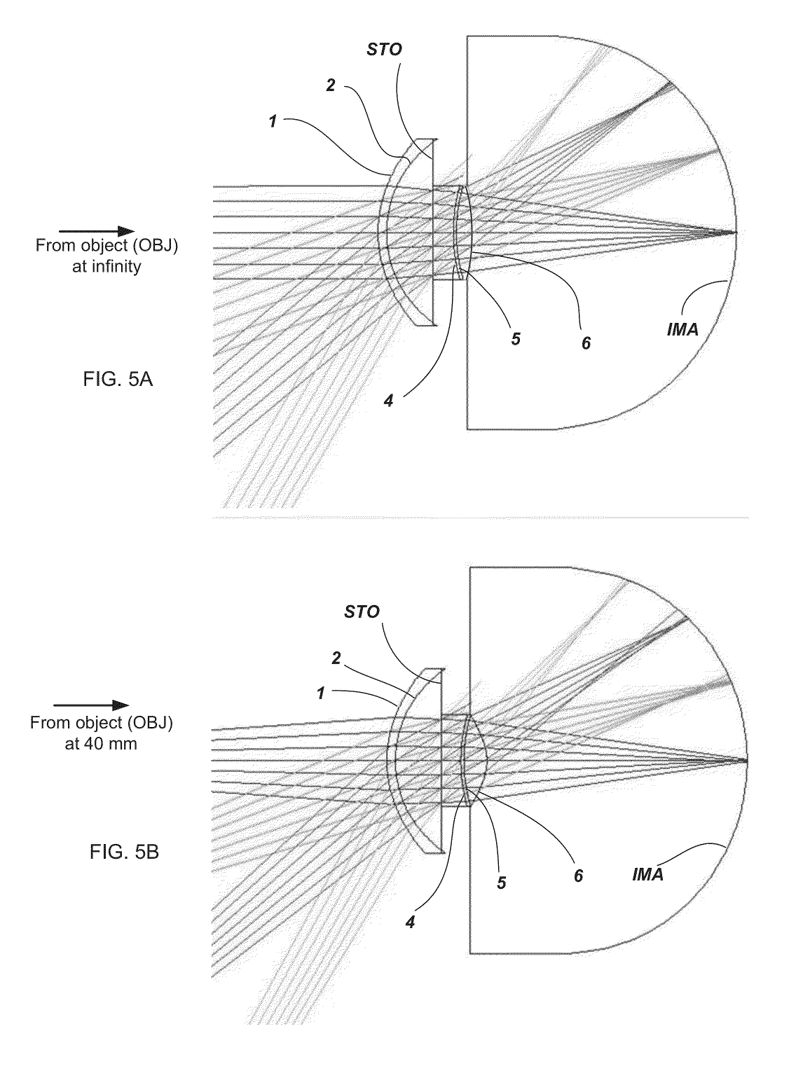

FIGS. 5A, 5B provide diagrams illustrating an optical layout used for raytracing of light through a model of an eye in which the natural lens is substituted with an embodiment of the IOL according to the invention from the object towards the retina to illustrate the ability of the embodiment of the invention to refocus within a dynamic range of distances (from infinity, corresponding to the layout of FIG. 5A, to about 40 mm, corresponding to the layout of FIG. 5B) substantially exceeding requirements that can be encountered in practice. Examples of Zemax® model design parameters corresponding to the layout of FIG. 5B are presented in Tables 1 and 2. The pupil stop was set for 5.1 mm (for accommodation at infinity) and 3 mm for near-distance accommodation. Surfaces 1, 2 represent the surfaces of the cornea; surface 3 (labelled as “STO”) corresponds to the aperture stop; surfaces 4, 5 correspond to the front outer or posterior surface 119 and the anteriorly disposed surface or boundary 114 of the IOL 116. Surface “IMA” corresponds to a surface of the retina.

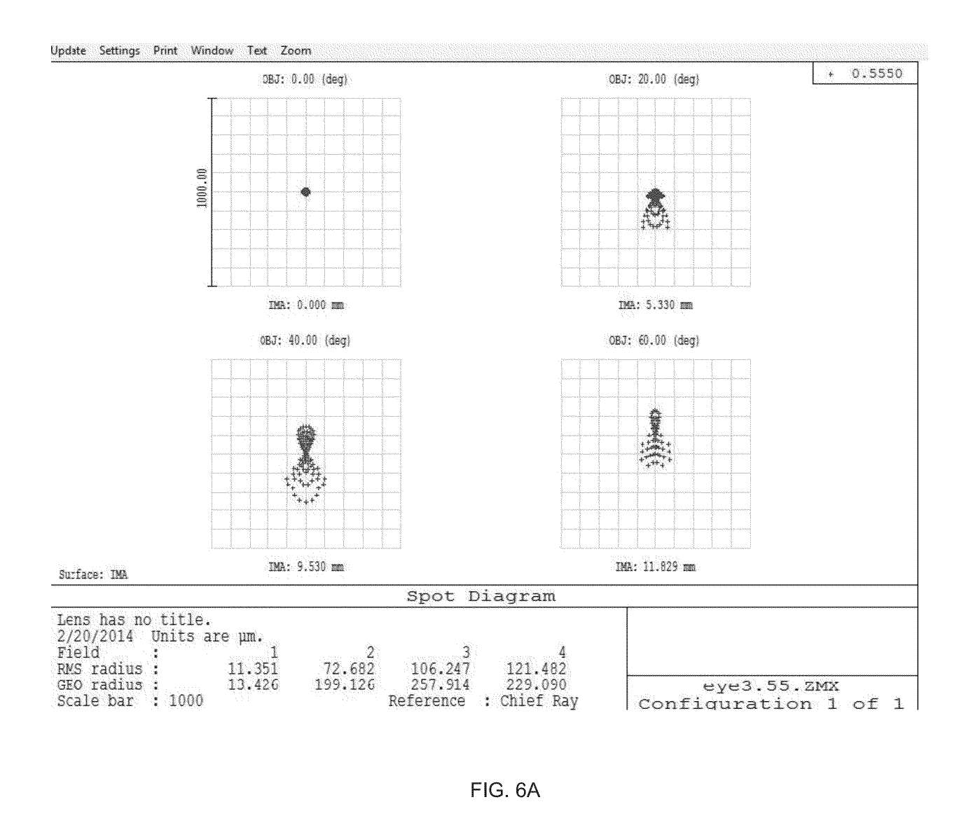

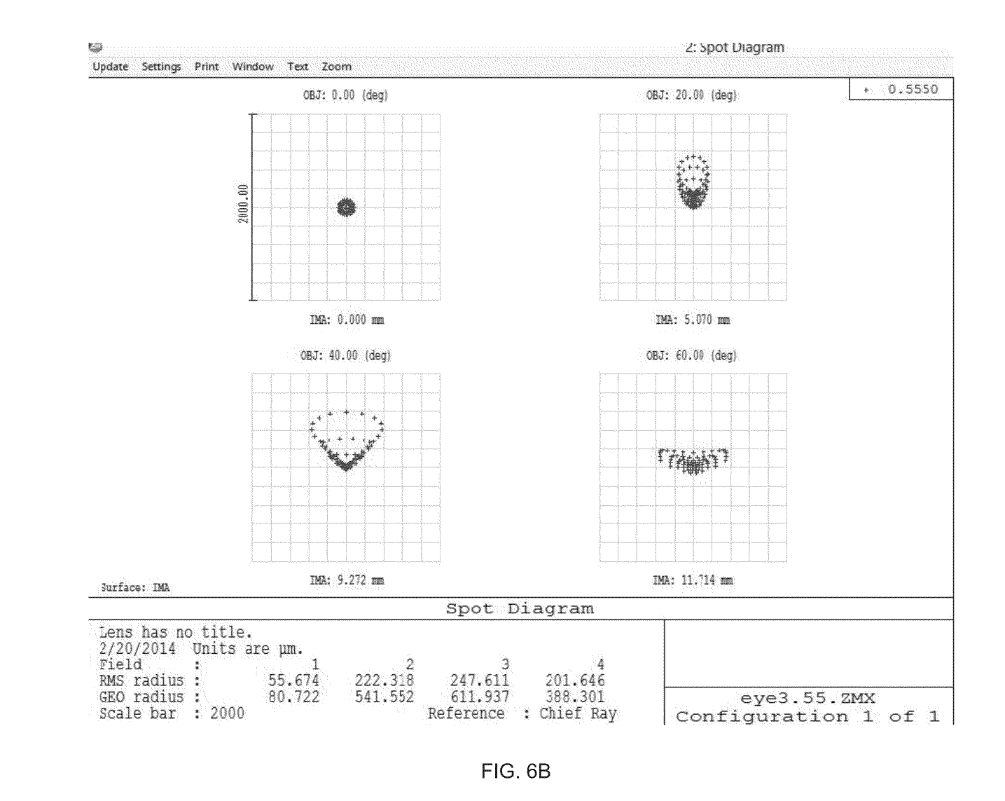

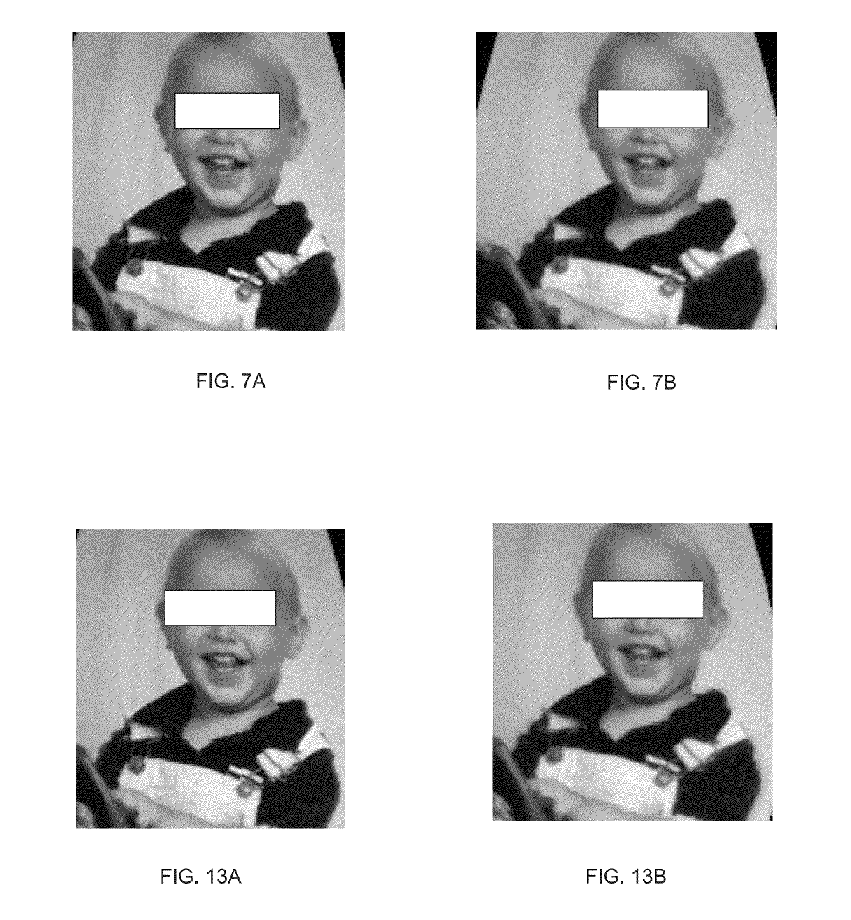

It is appreciated that the design for near/short distance accommodation was set to a 40 mm distance to object (FIG. 5B) to more clearly demonstrate a change of curvature of the prolate posterior aspheric surface 112 (shown as surface 6 in FIGS. 5A, 5b) when changing the accommodation of the IOL from the infinity to a near point source. In practice, as would be appreciated by a skilled artisan, the actual physiological design would be optimized for a near distance to object of about 200 mm or so. All design parameters summarized in Tables 1, 2 are initial estimates and not necessarily optimized and, therefore, corresponding spot diagrams (of FIGS. 6A, 6B) and simulated images (of FIGS. 7A, 7B) do not necessarily reflect the best quality of the imaging achievable with an embodiment of the IOL of the invention.

TABLE 1
Zemax ® design parameters corresponding to layout of FIG. 5A

| Surf: Type | | Comment | Radius | Thickness | Glass | Semi-Diameter | | Conic |
|---|---|---|---|---|---|---|---|---|
| OBJ | Standard | | Infinity | 1.000E+004 | | 1.733E+004 | U | 0.000 |
| 1* | Standard | | 7.800 | 0.550 | 377571 | 6.000 | U | −0.600 |
| 2* | Standard | | 7.000 | 2.970 | 337613 | 6.000 | U | −0.100 |
| STO | Standard | | Infinity | 1.300 | 337613 | 2.566 | U | 0.000 |
| 4* | Standard | | 11.000 | 0.200 | 500519 | 3.000 | U | 0.000 |
| 5* | Standard | | 11.000 | 1.000 | 500519 | 3.000 | U | 3.000 |
| 6* | Standard | | −16.100 | 16.950 | 336611 | 3.000 | U | −0.500 |
| IMA | Standard | | −13.400 | — | 336611 | 12.600 | U | 0.150 |

TABLE 2
Zemax ® design parameters corresponding to layout of FIG. 5B

| Surf: Type | | Comment | Radius | Thickness | Glass | Semi-Diameter | | Conic |
|---|---|---|---|---|---|---|---|---|
| OBJ | Standard | | Infinity | 40.000 | | 74.414 | U | 0.000 |
| 1* | Standard | | 7.800 | 0.550 | 377571 | 6.000 | U | −0.600 |
| 2* | Standard | | 7.000 | 2.970 | 337613 | 6.000 | U | −0.100 |
| STO | Standard | | Infinity | 1.300 | 337613 | 2.566 | U | 0.000 |
| 4* | Standard | | 11.000 | 0.200 | 500519 | 3.000 | U | 0.000 |
| 5* | Standard | | 11.000 | 1.500 | 500519 | 3.000 | U | 3.000 |
| 6* | Standard | | −3.100 | 16.950 | 336611 | 3.000 | U | −3.000 |
| IMA | Standard | | −13.400 | — | 336611 | 12.600 | U | 0.150 |

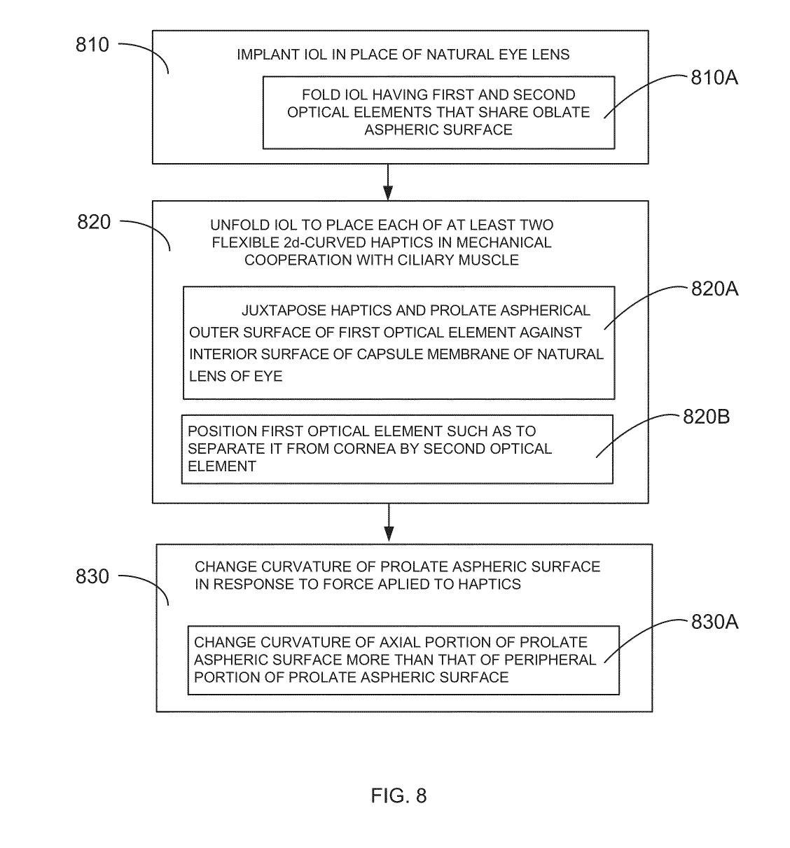

In reference to FIG. 8, the method for correcting vision includes implanting an IOL in an eye, at step 810, which IOL contains (i) a central optical portion that has an optical axis and that is formed by first and second optical elements that share an oblate aspheric surface, and (ii) at least two flexible curved haptics, each of said haptics having proximal and distal sides, the proximal side being integrated with the central optical portion along a perimeter thereof. The implantation may include folding the IOL, at step 810A. At step 820, so inserted IOL is unfolded inside the eye such as to place each of such 2D-curved haptics in mechanical cooperating with ciliary muscle of the eye. In particular, the step of unfolding may be associated with juxtaposing, at step 820A, said flexible haptics and said prolate aspherical surface of the first optical element against an interior surface of a capsule membrane of a natural lens of the eye such as to place distal side of each of said haptics in mechanical cooperation with the capsule membrane. The first optical element that has an outer prolate aspheric surface is placed, at step 820B, such as to be separated from the cornea by the second optical element. One of additional steps of the method may include step 830, during which a curvature of the prolate aspheric surface of the first optical element is changed, as a result of which a change of focal length of the IOL is realized. In particular, such change can be effectuated, at step 830A, to a higher degree in the axial portion of the prolate aspheric surface than in a peripheral portion of such surface.

According to a related embodiment of the invention, the problem of accommodating the focal length of an IOL is solved by applying a force mechanism supplied by the eye's ciliary muscle to an IOL structured to include two immediately-adjoining cells or chambers that are formed by outer wall elements (referred to herein as walls) and an internal flexible membrane. The flexible membrane is shared by the chambers is interchangeably referred to herein as an interior wall. The neighboring cells or chambers are filled with fluidic materials having different indices of refraction. For short, this embodiment may be referred to as a “fluidic IOL”. A posterior surface of this embodiment (at the outer wall of the posterior fluid cell) may be additionally reinforced to by a rigid optically transparent plate, which is in optical contact with such posterior surface and the shape of which remains substantially unchanged when the force from the ciliary muscle is passed onto the chamber(s) via flexible haptic(s) of the IOL. A principle of operation of this accommodating IOL, once it's installed in place of a natural eye lens, utilizes radial tension provided by relaxation of the ciliary muscle to create an anteriorly-vectored force on the IOL such as to allow the lens to alter the curvature of the internal flexible membrane and to cause a corresponding change in optical power characterizing at least one of the fluidic chambers.

Just like an embodiment described in reference to FIGS. 1A, 1B, the fluidic IOL is preferably azimuthally and radially homogeneous, such that within each material layer of the lens structure there are no discontinuities of material properties, shape, or refractive index. Such continuity of material and geometrical properties allows to minimize glare, caused by light scatter and diffractive effects in operation, once the fluidic IOL has been installed in the eye instead of the natural lens.

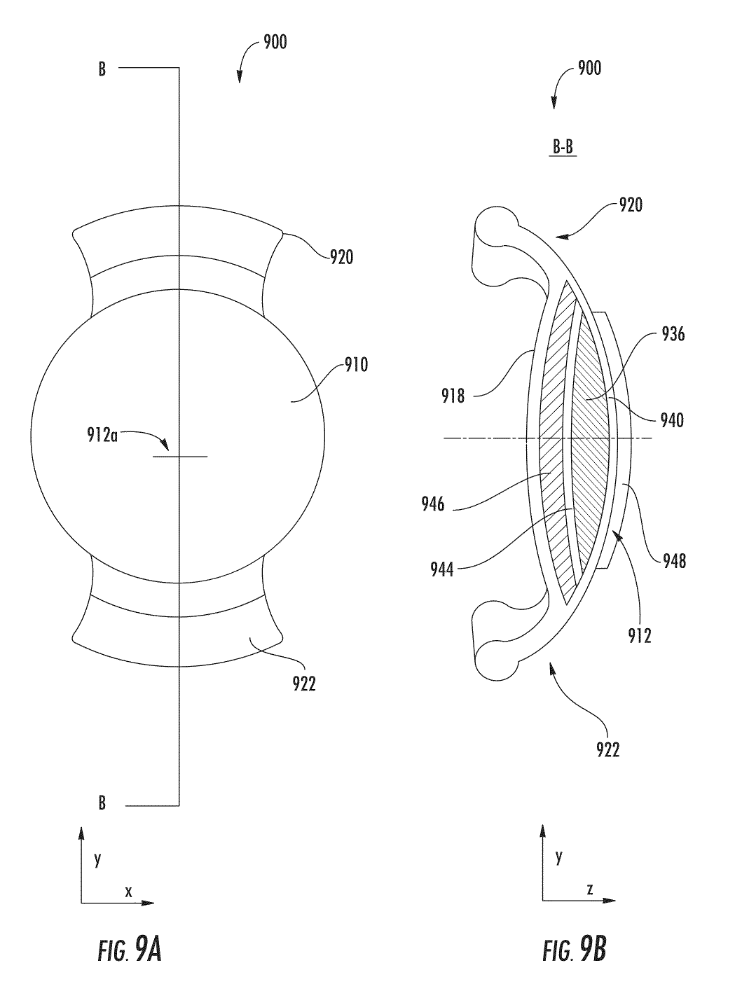

FIGS. 9A, 9B illustrate schematically an embodiment 900 of the fluidic IOL according to the invention, in front view and in a cross-sectional view, respectively. The local system of coordinates is chosen such that the z-axis generally corresponds to a direction of ambient light propagation through the IOL that has been implanted in the eye.

The embodiment 900 includes an optical portion 910 containing a first, posterior lenticle or lenslet 936, defined by a posterior chamber formed by an outer wall or layer 940 and an internal flexible membrane 944. The optical portion 910 additionally includes a second, anterior lenticle or lenslet 946 defined by an anterior chamber formed by the internal flexible membrane 944 and a stabilizing plate (or outer wall or layer) 918 corresponding to the haptic portion of the IOL 900. The perimeter of the interior flexible membrane is integrated with and/or affixed to the peripheral portions of the walls 940, 918 such that the flexible membrane 944 bisects the space between the walls 918, 940 to substantially completely define spatial separation between contents of the anterior and posterior chambers, without the use of any additional rigid chamber-separating portion. The spacings between the housing elements 918, 940, 944 that form the lenslets 936, 946 are filled with fluids, such that the fluid in the anterior chamber has an index of refraction that is higher than that of the fluid in the posterior chamber by, for example, 0.1. Generally, the fluidic materials used in lenslets 936, 940 have refractive indices within the range from about 1.38 and 1.55, with the difference of these refractive indices having a value within the range from about 0.05 and about 0.2.

Non-limiting examples of such fluids are provided by silicon oils and glycerin.

The haptic portion, in addition to the stabilizing plate 918 may include haptic wing(s) 920, 922 between which the plate 918 continuously extends. (In a specific implementation, the stabilizing plate 918 may be structured as a lenslet possessing optical power, for example by analogy with a specific implementation of the element 118 of FIG. 1A.) Generally, the overall haptic portion(s) of the IOL 900 is similar in structure to the haptic portion(s) discussed in reference to FIGS. 1A, 1B, and 4, possesses material and operational characteristics as discussed above, and for that reason will not be described here in any more detail. Additional and/or alternative details of structure of haptic(s) for the IOL are discussed in a co-pending application PCT/US13/55093, the disclosure of which is incorporated herein by reference in its entirety for all purposes. To the extent that any inconsistency or conflict exists in a definition or use of a term between a document incorporated herein by reference and that in the present disclosure, the definition or use of the term in the present disclosure shall prevail.

It is appreciated that in the case of one specific implementation, the embodiment 900 is structured as a bicameral chamber housed by the semi-rigid walls 918, 940 (that are connected along their respective perimeters and made, for example, from an acrylic material, the constituent sub-chambers of which are separated by the internal (intracameral) flexible membrane 944. The outer wall 940 may be additionally re-enforced by the rigid, optically transparent plate 948 that is substantially congruent with the wall 940 at least in the central, optically operational portion of the lens 900. While generally materials used for construction of the (semi-) rigid plates of an embodiment may differ to optimize the opto-mechanical operational characteristics of a particular embodiment (as a person of ordinary skill in the art will readily understand), in one specific case the outer shell walls of the lens 900 may be made of standard usage foldable acrylic, while the internal flexible membrane may be made of silicone.

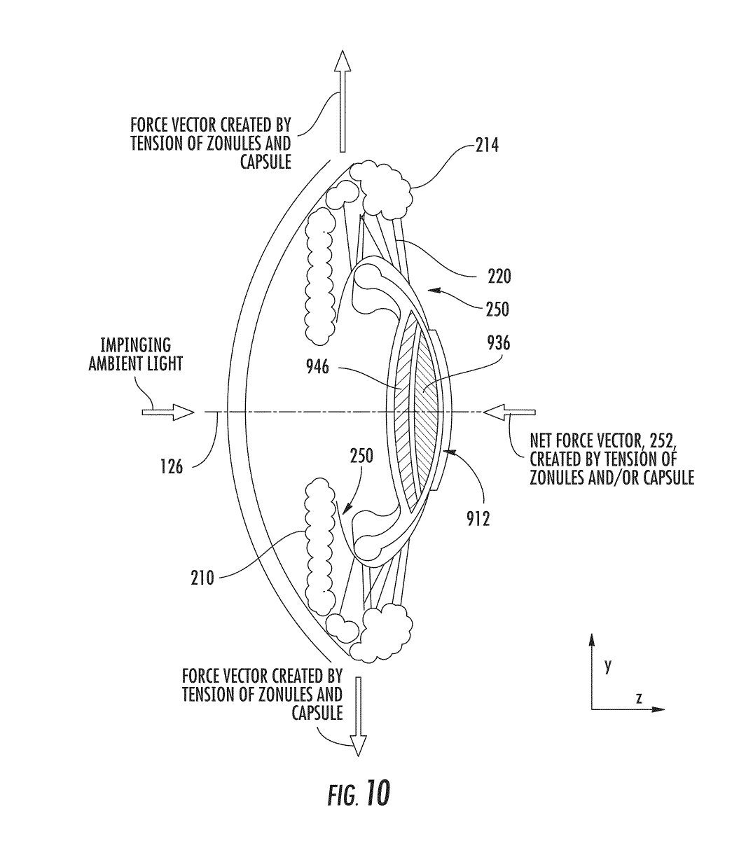

In operation, the embodiment 900 is installed behind the cornea in a fashion similar to that described in reference to FIG. 2B or FIG. 2C. Referring now to FIG. 10, which schematically illustrates an example of positioning of the IOL 900 instead of the natural lens of the eye—in this case, within the lens capsule 250—and in further reference to FIG. 9A, the IOL 900 (whether equipped or not equipped with the reinforcing plate 948) is placed within the capsule membrane 1050 of the natural lens of the eye such that flexible haptic(s) 920, 922 and the outer surface of the wall 940 are spatially conforming to the capsule membrane 250 and such as to place distal side of the haptic(s) in mechanical cooperation with the internal side of the capsule membrane. In an unstressed state, the shape of a posterior surface 912 of the wall 940 is that of a prolate asphere having an apex 912a. The curved shape of the haptic(s) is structured to maximally transfer the pressure applied by the capsule membrane to the posterior surface of the bicameral chamber of the IOL 900 and to conform to native shape of the natural lens of the eye that is being replaced, which facilitates maintaining the capsule membrane in its physiological shape to allow for accommodation of the preudophakic lens implant.

The change in opto-geometrical parameters of the lens 900 is caused, in operation, in a fashion similar to that described above with respect to the embodiment 100, by patient's focusing on an object at any given distance within the field-of-view with an autonomic nervous system feedback response. When the ciliary muscle 214 is relaxed (during a distance focusing of the eye), tension is increased on the zonules 220 and the lens capsule 250, similar to the tightening of a drum head. Increasing tension on the lens capsule applies an anteriorly directed force 252 on the capsular membrane 250 and displaces the capsular membrane posteriorly. This movement transfers pressure from the capsular membrane to the posterior surface 912 of the lenticle 936 and anteriorly displaces the posterior lenticle 936 acting as a piston to pressurize the posterior chamber and spherically deform the interior flexible membrane 944 anteriorly. A skilled artisan will readily understand that, due to the differences between the refractive indices of the fluid contents of the lenslets 936, 946, with such deformation and/or repositioning of the membrane 944 and while the walls 918, 940 remain substantially unchanged, the effective optical power of the whole lens 900 is decreased in proportionately (in a specific case—in direct proportion) to the posteriorly-applied pressure. As the flexible membrane 944 is present across the whole clear aperture of the lens 900 (it is affixed internally to the perimeter edge of the outer shell of the lens), the produced change in the effective optical power is substantially the same at any point within the clear aperture of the lens 900.

Such change of the power of the overall lens is accompanied by a change of optical power of at least one of the constituent lenslets 936, 946. Optically, this effect is equivalent to relaxation of the natural lens in an eye that accommodates to focus on a distant object. Conversely, during the accommodation on a near-by object, the ciliary muscle 214 contracts, relaxing the tension on the zonules 220 and/or the capsular membrane 250. The relaxed tension decreases the pressure on the posterior surface 912, allowing it to resume its unstressed shape. This in effect increases the power of the lens 900 just as the natural lens does during accommodation to focus on a near object.

It is appreciated that material composition of IOL embodiments of the invention allows the IOLs to be folded and inserted into the eye through a small incision (which make them a better choice for patients who have a history of uveitis and/or have diabetic retinopathy requiring vitrectomy with replacement by silicone oil or are at high risk of retinal detachment). In the case of IOL 900, for example, it implies that at least one of (i) semi-rigid spatially-continuous haptic(s) 920, 922 integrated with the anterior stabilizing plate 918 along its edge and (ii) the posterior wall 940 and/or plate 948 are structured to be appropriately foldable and/or bendable.

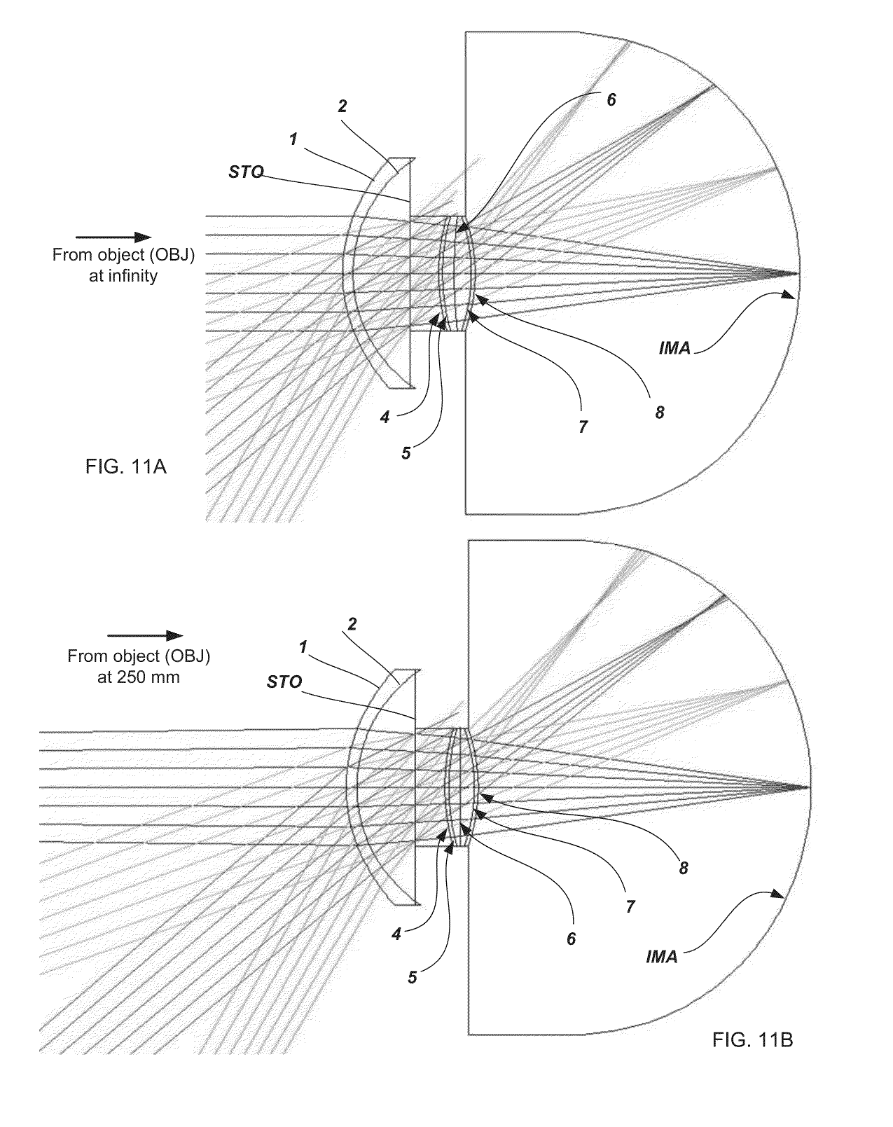

FIGS. 11A, 11B provide diagrams illustrating an optical layout used for raytracing of light through a model of an eye in which the natural lens is substituted with an embodiment of the IOL according to the invention from the object towards the retina to illustrate the ability of the embodiment of the invention to refocus within a dynamic range of distances (from infinity, corresponding to the layout of FIG. 11A, to about 250 mm, corresponding to the layout of FIG. 11B) thereby easily satisfying requirements that can be encountered in practice. Examples of Zemax® model design parameters corresponding to the layouts of FIGS. 11A, 1B are presented in Tables 3 and 4. As customary in Zemax®, the geometrical dimensions are provided in millimeters. The pupil stop was set for 5.1 mm (for accommodation at infinity) and 3 mm for near-distance accommodation. Surfaces 1, 2 represent the surfaces of the cornea; surface 3 (labelled as “STO”) corresponds to the aperture stop; surfaces 4, 5 correspond to the posterior and anterior surfaces of the plate 118, surface 6 represents the internal flexible membrane 944, surfaces 7, 8 correspond to the posterior and anterior surfaces of the anterior wall 940. Surface “IMA” corresponds to a surface of the retina.

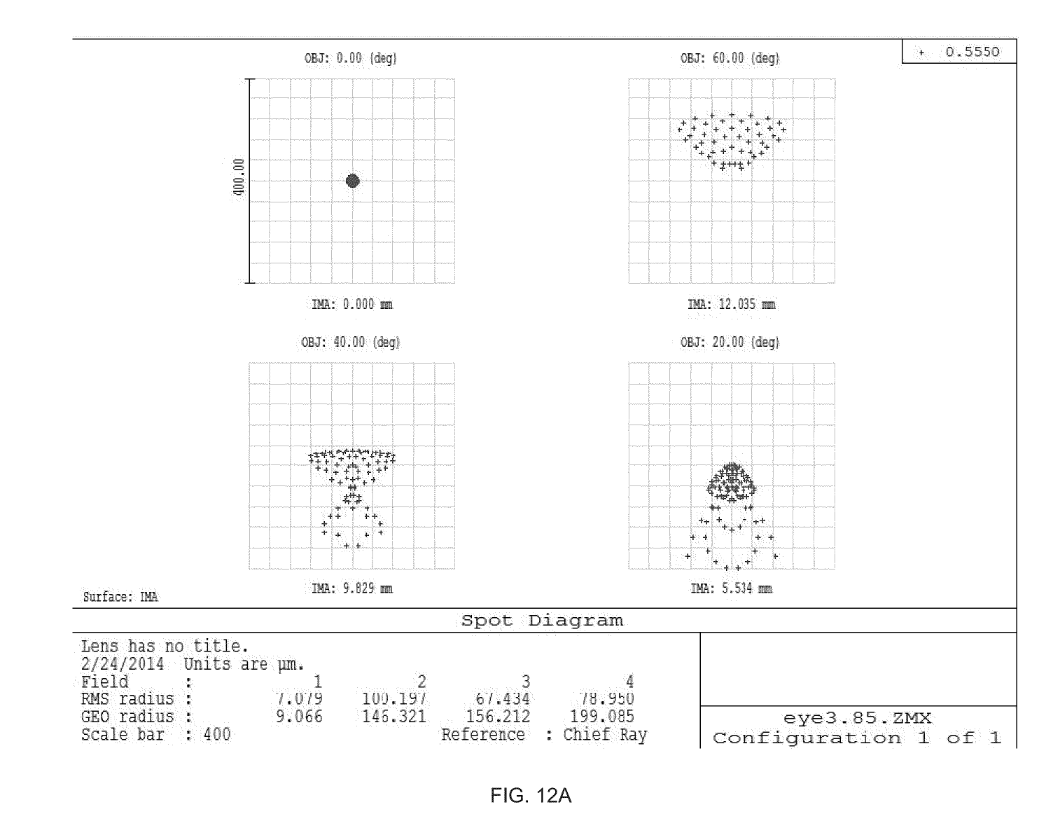

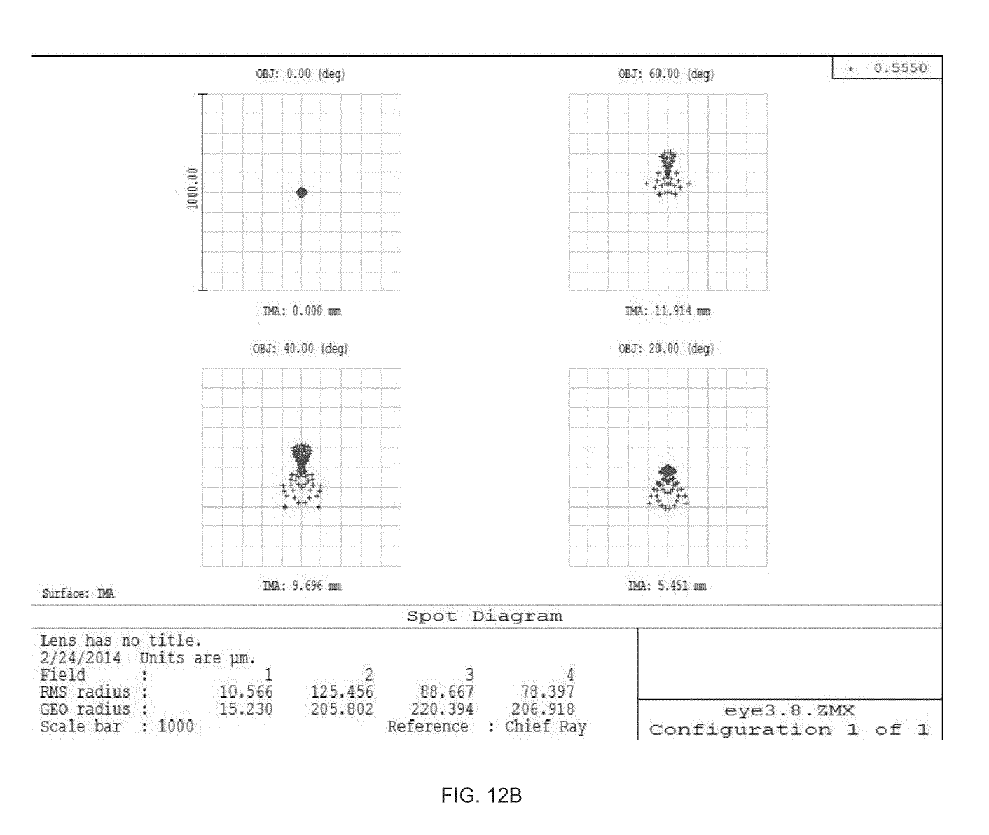

It is appreciated that for the purposes of demonstration of practicality of the proposed design, the design for near/short distance accommodation was set to a 250 mm distance between the lens 900 and the object. The design parameters in Tables 3 and 4, and evidence the effect of the curvature of the flexible membrane on the optical power of the embodiment. These parameters used for the presented operation of lens 900 are not necessarily optimized and, therefore, corresponding spot diagrams (of FIGS. 12A, 12B) and simulated images (of FIGS. 13A, 13B) do not necessarily reflect the best quality of the imaging achievable with an embodiment of the IOL of the invention.

TABLE 3
Zemax ® design parameters corresponding to layout of FIG. 11A

| Surf: Type | | Comment | Radius | Thickness | Glass | Semi-Diameter | | Conic |
|---|---|---|---|---|---|---|---|---|
| OBJ | Standard | | Infinity | 250.000 | | 438.145 | U | 0.000 |
| 1* | Standard | | 7.800 | 0.550 | 377571 | 6.000 | U | −0.600 |
| 2* | Standard | | 7.000 | 2.970 | 337613 | 6.000 | U | −0.100 |
| STO* | Standard | | Infinity | 1.500 | 337613 | 2.800 | U | 0.000 |
| 4* | Standard | | 11.100 | 0.200 | 525519 | 3.000 | U | 1.500 |
| 5* | Standard | | 11.100 | 0.600 | 535519 | 3.000 | U | 1.500 |
| 6* | Standard | | Infinity | 0.700 | 415519 | 3.000 | U | 0.000 |
| 7* | Standard | | −8.500 | 0.200 | 525519 | 3.000 | U | −2.000 |
| 8* | Standard | | −8.500 | 16.930 | 336611 | 3.000 | U | −2.000 |
| IMA | Standard | | −13.400 | — | 336611 | 12.600 | U | 0.150 |

TABLE 4
Zemax ® design parameters corresponding to layout of FIG. 11B

| Surf: Type | | Comment | Radius | Thickness | Glass | Semi-Diameter | | Conic |
|---|---|---|---|---|---|---|---|---|
| OBJ | Standard | | Infinity | 1000.000 | | 1737.183 | U | 0.000 |
| 1* | Standard | | 7.800 | 0.550 | 377571 | 6.000 | U | −0.600 |
| 2* | Standard | | 7.000 | 2.970 | 337613 | 6.000 | U | −0.100 |
| STO* | Standard | | Infinity | 1.500 | 337613 | 3.000 | U | 0.000 |
| 4* | Standard | | 11.100 | 0.200 | 525519 | 3.000 | U | 1.500 |
| 5* | Standard | | 11.100 | 0.600 | 535519 | 3.000 | U | 1.500 |
| 6* | Standard | | 26.000 | 0.900 | 415519 | 3.000 | U | 0.000 |
| 7* | Standard | | −8.500 | 0.200 | 525519 | 3.000 | | −2.000 |
| 8* | Standard | | −8.500 | 16.930 | 336611 | 3.000 | | −2.000 |
| IMA | Standard | | −13.400 | — | 336611 | 12.600 | U | 0.150 |

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

In addition, it is to be understood that no single drawing is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

The invention as recited in claims appended to this disclosure is intended to be assessed in light of the disclosure as a whole. Disclosed aspects, or portions of these aspects, may be combined in ways not listed above. Accordingly, the invention is not intended and should not be viewed as being limited to the disclosed embodiment(s).

1. A pseudophakic lens comprising:
a first rotationally symmetric optical portion having an optical axis and a first optical power,
wherein a first volume of said first rotationally symmetric optical portion is defined by a posterior curved plate having a first perimeter and a flexible membrane,
said first volume filled with a first fluid having a first refractive index;
a second rotationally-symmetric optical portion co-axial with the first rotationally symmetric portion and having a second optical power,
wherein a second volume of said second rotationally symmetric optical portion is defined by an anterior rigid curved plate having a second perimeter and said flexible membrane,
said second volume filled with a second fluid having a second refractive index;
said posterior and anterior plates being integrated with one another along said first and second perimeters;
said flexible membrane being sealingly affixed to at least one of said posterior and anterior plates at at least one of the first and second perimeters such as to prevent dispensation of any of the first and second fluids from a respectively corresponding volume of the first and second volumes; and
first and second flexible haptic wings, each having proximal and distal sides, the proximal side being integrated with at least the anterior plate at least along the first perimeter;
said first and second optical portions being operable to gradually change at least one of the first and second optical powers in response to deformation of said membrane while the anterior and posterior plates substantially maintain their corresponding shapes.

2. A pseudophakic lens according to claim 1, said lens being dimensioned to be placed, in operation, in mechanical cooperation with a ciliary body muscle of an eye of a subject such that, in response to tension applied to at least one of zonules and capsular membrane of a natural lens of the eye by the ciliary body muscle, an anteriorly-vectored force is administered to said posterior plate, causing deformation of said membrane by transferring of pressure thereto from the posterior plate through the second fluid.

3. A pseudophakic lens according to claim 2, wherein said deformation is spherical and caused substantially without axial repositioning of any of the posterior and anterior plates.

4. A pseudophakic lens according to claim 1, wherein a surface of the posterior plate in unstressed state is prolate aspheric.

5. A pseudophakic lens according to claim 1, wherein said lens is dimensioned to be placed, during an implantation of said lens in an eye, inside a capsular membrane of the natural lens of the eye and wherein each of said haptic wings is curved to conform to a shape of said capsular membrane.

6. A pseudophakic lens according to claim 1, wherein said lens is dimensioned to enable positioning of a distal side of each of said haptic wings, during an implantation of said lens in an eye, in a sulcus between a root of the iris of the eye and ciliary body muscle of the eye.

7. A pseudophakic lens according to claim 1, further comprising a rotationally symmetric stabilizing plate made from an optically transparent material, said stabilizing plate having a surface congruent with that of said posterior plate, said stabilizing plate being integrated with said posterior plate along an outer surface thereof.

8. A pseudophakic lens having an optical power and comprising:
a bicameral chamber defined by rigid and foldable anterior and posterior curved layers of material integrated with one another along corresponding perimeters thereof;
a flexible and deformable membrane disposed between said anterior and posterior layers to form first and second cameras, of said chamber, filled respectively with first and second fluids having different indices of refraction;
said membrane being sealingly and directly affixed to said corresponding perimeters to prevent leakage of any of said first and second fluids from corresponding cameras;
the lens being operable to transfer pressure, applied anteriorly to said posterior layer, to the membrane such as to change the optical power in response to spherical deformation of membrane caused by said transfer.

9. A pseudophakic lens according to claim 8, wherein the lens is operable to change the optical power in response to said deformation while said deformation is accompanied by at least one of (i) the anterior and posterior layers substantially maintaining their corresponding shapes, and (ii) the anterior and posterior layers substantially maintaining their corresponding axial positions.

10. A pseudophakic lens according to claim 8, wherein a surface of the posterior plate in unstressed state is prolate aspheric.

11. A presudophakic lens according to claim 8, further comprising first and second flexible haptic wings, each having proximal and distal sides, the proximal sides being integrated with at least the anterior layer at least along perimeter thereof.

12. A pseudophakic lens according to claim 11, wherein said lens is dimensioned to be placed, during an implantation of said lens in an eye, inside a capsular membrane of the natural lens of the eye such that each of said haptic wings is curved to conform to a shape of said capsular membrane.

13. A method for correcting vision with the use of an intraocular lens (IOL), the method comprising:
implanting the IOL in an eye of the patient, the IOL having
a bicameral chamber defined by rigid and foldable anterior and posterior curved layers of material integrated with one another along corresponding perimeters thereof;
a flexible and deformable membrane disposed between said anterior and posterior layers to form first and second cameras, of said chamber, filled respectively with first and second fluids having difference indices of refraction; and
first and second flexible haptic wings, each having proximal and distal sides, the proximal sides being integrated with at least the anterior layer at least along perimeter thereof;
said membrane being sealingly and directly affixed to said corresponding perimeters to prevent leakage of any of said first and second fluids from corresponding chambers;
the lens being operable to transfer pressure, applied anteriorly to said posterior layer, to the membrane such as to change the optical power in response to spherical deformation of membrane caused by said transfer,
and
juxtaposing said haptic wings and said posterior layer against an interior surface of a capsule membrane of a natural lens of the eye such as to place distal side of each of said haptic wings in mechanical cooperation with said capsule membrane.

14. A method according to claim 13, further comprising spherically deforming said flexible membrane by applying, to the posterior layer, force directed anteriorly.

15. A method according to claim 14, wherein said spherically deforming includes applying force cause by flexing of the ciliary muscle of the eye.

16. A method according to claim 13, further comprising changing optical power of said IOL by deforming said flexible membrane due to compression of said first fluid while maintaining respective axial positions and shapes of said posterior and anterior layers.

17. A method according to claim 13, wherein said implanting includes folding the anterior layer said juxtaposing includes unfolding the anterior layer.
